(19) United States
(12) Patent Application Publication
(10) Pub. No.: US 2009/0259089 A1
(43) Pub. Date: Oct. 15, 2009

(54) Expandable catheter for delivery of fluids

(76) Inventors: Daniel Gelbart, Vancouver (CA); Samuel Victor Lichtenstein, Vancouver (CA)

Correspondence Address: Daniel Gelbart, 4706 DRUMMOND DR, VANCOUVER, BC, V6T-1B4, CA

(21) Appl. No.: 12/222,134

(22) Filed: Aug. 4, 2008

Related U.S. Application Data
(63) Continuation of application No. 12/081,050, filed on Apr. 10, 2008, now abandoned.

(51) Int. Cl. A61M 1/12; A61M 25/10

(52) U.S. Cl. 600/16; 604/103.01

(57) ABSTRACT

A multi lumen catheter is made from a number of thin walled flexible tubes bonded together to form an inner lumen. The inner lumen can withstand vacuum when the outside tubes are pressurized. During insertion the tubes are compressed and collapsed. The tubes expand by the pressure of the pumped fluid. At the point the catheter enters the body the expansion is restricted to a smaller diameter than the rest of the catheter.

### FIELD OF THE INVENTION

The invention is in the medical field, and is particularly useful for minimally invasive surgery such as used with ventricular assist pumps.

### BACKGROUND OF THE INVENTION

In many minimally invasive surgical procedures a catheter is inserted into the body. Traditionally the opening required to introduce the catheter has to be the size of the catheter outside diameter. For applications requiring insertion of rigid or semi-flexible items, such as stents, the inner diameter of the catheter has to accommodate the inserted item at all points along the catheter. This determines the outside diameter. This is not required for the delivery of fluids, as fluids can adapt to a varying cross section. The resistance to the flow of a fluid is determined by the sum of all the resistances the fluid encounters. It is possible to have a high flow rate in a catheter of a variable inside diameter, as long as the sections having a smaller diameter are very short and the transitions between the different diameters is smooth and conducive to good flow characteristics. The invention takes advantage of this property to allow a large flow in a catheter that can be inserted into the body through a small incision. The invention is particularly useful in devices known as Cardiac Assist Devices, external Artificial Hearts or Ventricular Assist Devices (VADs for short). Such devices are used to help, or fully replace, the function of the heart. Normally they are used for short periods, days to weeks, but in some cases they can be used as an external artificial hearts for life long support. In VADs the required flow rates are large, in the order of 5 l/min of blood, while the incision into the artery has to be minimized. Blood can tolerate a limited range of pressure and vacuum, therefore the flow can not be increased simply by increasing pressure or suction. In general, blood should not be exposed to pressures of more than 800 mmHg above atmospheric or suction stronger than −400 nnHg. High shear rates should be avoided as well. High shear rates can occur when valves are used, as narrow slots are created momentarily as valve closes. Valves are also susceptible to clotting and mechanical failure. Some prior art VAD use expensive miniature turbines to avoid valves, but since the turbine is coming in contact with the blood it is disposed after each procedure, a very costly procedure. Other disposable VADs use valves, which are expensive and increase the size of required incision in order to introduce the catheter. It is an object of the present invention to have high flow rates via a small incision. Another object is to have a VAD devices that in very compatible with the ideal conditions for handling blood. A further object is to have a VAD with all the parts coming into contact with the blood being of a low cost disposable type. Further advantages will become apparent from studying the disclosure and the drawings.

### SUMMARY OF THE INVENTION

A multi lumen catheter is made from a number of thin walled flexible tubes bonded together to form an inner lumen. The inner lumen can withstand vacuum when the outside tubes are pressurized. During insertion the tubes are compressed and collapsed. The tubes expand by the pressure of the pumped fluid. At the point the catheter enters the body the expansion is restricted to a smaller diameter than the rest of the catheter.

### BRIEF DESCRIPTION OF THE DRAWINGS

FIG. 1 is a general view of a catheter with a single collapsible tube as used in a VAD application.

FIG. 2 is a section of the catheter inserted into a body lumen.

FIG. 3 is a perspective view of the catheter inserted into a body lumen.

FIG. 4 is a section showing the distal part of the catheter inserted into the left ventricle via the aorta in a VAD application.

FIG. 5 is a cross section of the catheter at the area it enters the body.

FIG. 6 is a general view of a catheter having both suction and pressure lumens collapsible.

FIG. 7A is a cross section of the catheter of FIG. 6.

FIG. 7B is an alternate embodiment of a catheter having both suction and pressure lumens collapsible.

FIG. 8 is a general view of the distal part of the catheter of FIG. 6.

FIG. 9 is a general view of an alternate blood pump.

### DETAILED DESCRIPTION

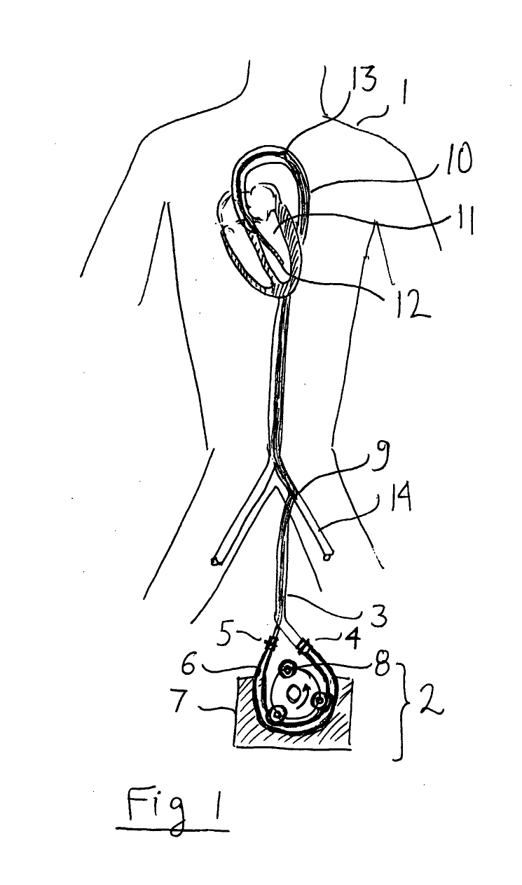

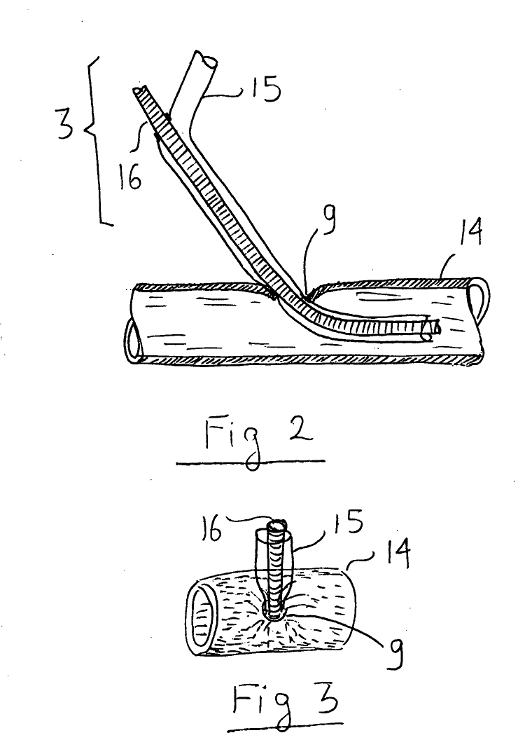

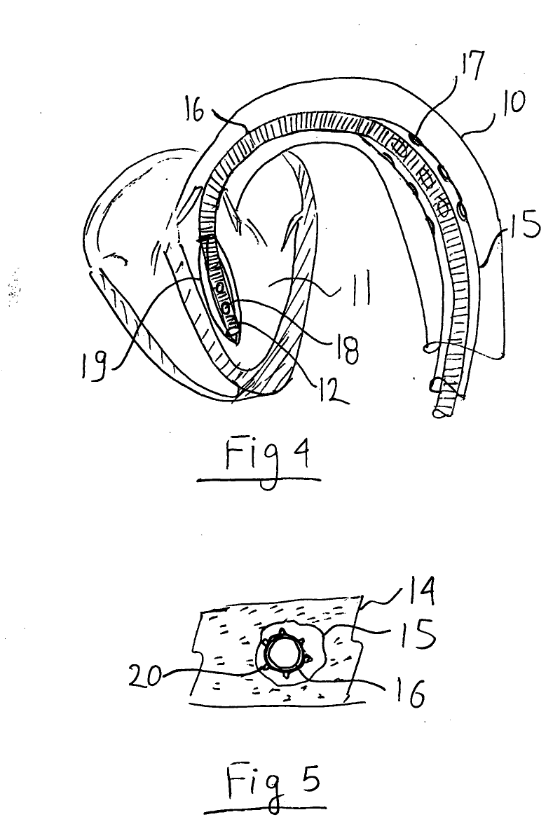

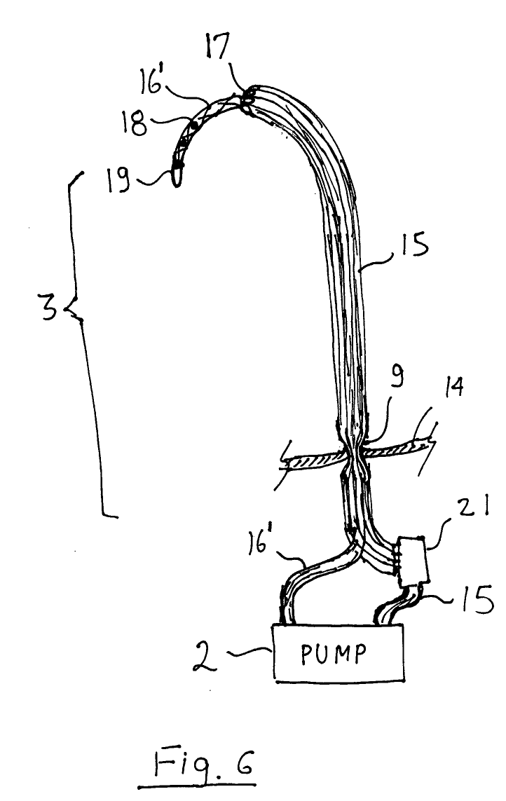

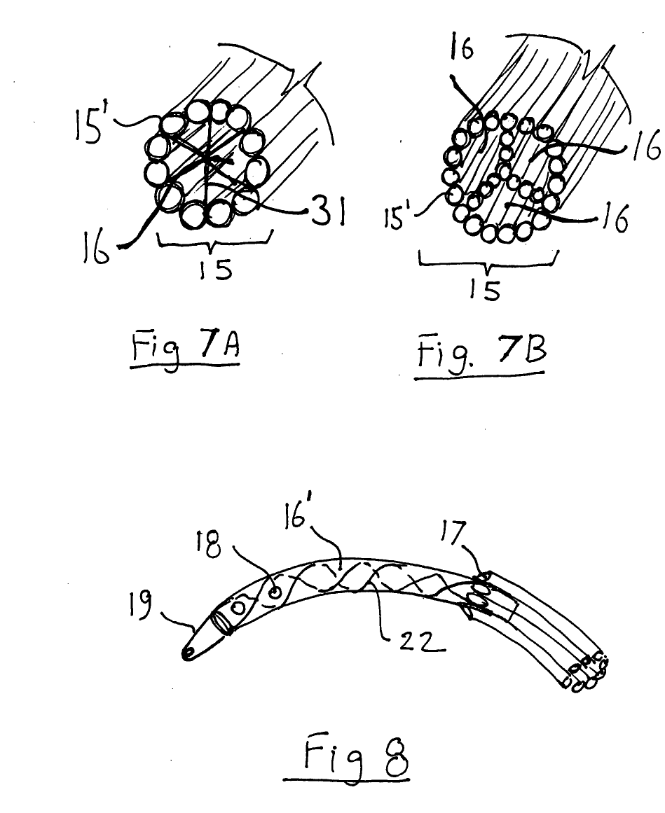

1. A catheter for cardiac assist devices such as VADs is shown in FIG. 1. The patient 1 is connected to an external pump 3 via catheter 3. Catheter 3 is normally connected to pump 2 via connectors 4 and 5. The catheter comprises of a flexible inner tube 16, shown in FIG. 2, and a collapsible outer sleeve 15, shown in FIG. 2. Sleeve 15 is typically more flexible and of thinner material than inner tube 16. It is inserted into aorta 10 via point of entry 9, typically an incision or opening at the femoral artery 14. The distal end 13 of the outer sleeve is typically discharging blood into the aorta 10 while the distal end 12 of the inner tube is typically taking blood out of the left ventricle 11. Pump 2 can be a peristaltic pump, as shown in FIG. 1, a bag based pump as shown in FIG. 9, a centrifuigal pump, a turbine pump or even a bellows or piston pump. In a peristaltic pump, a flexible hose 6 is being compresses by at least two revolving rollers 8 against a block 7. Since blood can be damaged by high shear forces, tube 6 of pump 2 should have a significantly higher diameter than sleeve 15. This allows rollers 8 to rotate slowly, minimizing sheer and increasing pump life. If desired, the compression can leave a small gap in the tube, trading off pumping efficiency with damage to blood cells. Valveless pumps are believed to cause less damage to blood and sometimes less clotting. Pumps for pumping blood are commercially available devices used in open heart surgery and artificial hearts and need not be detailed here. Some well known blood pumps are Medtronics Bio-Pump, Ambiomed Impella, DeBakey VAD and others. In order to minimize size of opening 9 in artery the suction and delivery pressure should be as high as compatible with blood handling. Typical suction (vacuum) is −400 to −600 mmHg but higher suction, up to 700 mmHg can be used at times. The delivery pressure to the outside sleeve can be chosen over a wide range, but in order to achieve high flow it is desired to select a pressure over 500 mmHg, typically 750 mmHg. Higher pressure, as high as 2500 mmHg can be used but are limited by strength of expandable sleeve. For other fluids these restriction normally do not apply. All the stated pressures are relative to atmospheric pressure. The catheter can be made from common materials used for catheters, such as PVC, Nylon or Polyethylene. It is sometimes desired to reinforce the inner tube with a metal spring in order to prevent collapse because of the vacuum used for suction. The outer sleeve 15 should be made very thin and flexible, in order to collapse around the inner tube when catheter is inserted into a lumen such as artery. This is shown in FIGS. 2 and 3. When catheter 3 is inserted into artery 14 the outer sleeve is compressed at the point of entry 9. When sleeve 2 is pressurized it will fully expands along its whole length inside and outside the body, and partially expand at point 9. This entry point increases the resistance to flow, but greatly reduces the size of hole in artery. The increased resistance is compensated by higher pressure at pump output. Because relatively high vacuum and pressure is used (compared to regular blood pumps), centrifugal pumps, such as the Medtronics Bio-Pump, need to be run at higher speeds than conventionally run or two pumps can be connected in series. Performance of these pumps increases dramatically with motor speed. By the way of example, an inner tube of 6 mm diameter and an outer sleeve made of 0.1 mm polyethylene expanded to 12 mm inside the body, but limited to about 8 mm at entry point 9 can pump about 5 l/min of blood when used with −400 mm suction and 750 mm of pressure. A typical range of diameters is the outside diameter of the catheter at the point of insertion is between 3 to 8 mm and the diameter of the catheter inside the body is between 5 and 15 mm.

If desired the flow can be pulsed rather than continuous, but this normally reduces throughput. The advantage of pulsed flow is mimicking the natural action of the heart. The soft sleeve adapts to the shape of the hole in the artery and causes much less trauma than regular catheters of same size. Referring now to FIG. 4, the distal end of sleeve 15 has discharge ports 17, typically discharging into aorta 10. The distal end 12 of the inner tube 16 is typically located in the left ventricle 11. Blood is sucked into tube via ports 18. A collapsible wire cage 19, made of Nitinol, stainless steel or a polymer can be used to prevent the suction from causing the tissue to block the ports 18. The cage is collapsible in order to pass through the small entry point to the body. Because the outer sleeve 15 maybe fully compressed onto the inner tube by the forces at the entry point, it may be desired to provide a texture on the outside of the inner tune, at least at the point of entry, to prevent blocking the flow. This is shown in FIG. 5. Inner tube 15 has ribs or other protrusions 20 in order to leave a passage for the blood even if sleeve 15 is compressed against the inner tube.

An even greater reduction in the size of the required entry hole can be achieved with the catheter design shown in FIG. 6. The inner tube (suction tube) is eliminated and replaced by the lumen created by bonding together a group of outer tubes (pressure tubes) to form a shape that, when pressurized, can support a vacuum applied to the central space. Two such possible shapes are shown in FIG. 7A and 7B. Pressure tube 15 is made up of multiple tubes 15′ bonded together to form a lumen 16 for the suction. When tubes 15′ are not pressurized the whole assembly can be collapsed and compressed to a very small cross section. The number of tubes 15′ can vary from 3 to over 30 and many layout patterns can be arranged to support vacuum at the center. A combination of different diameter tubing can also be used. The pressure has to be applied before the vacuum. As soon the pressure is applied tube 15 expands and can support vacuum in the central lumen 16. The central lumen can be further divided by partitions 31 to decrease the turbulence, decreasing hemolysis.

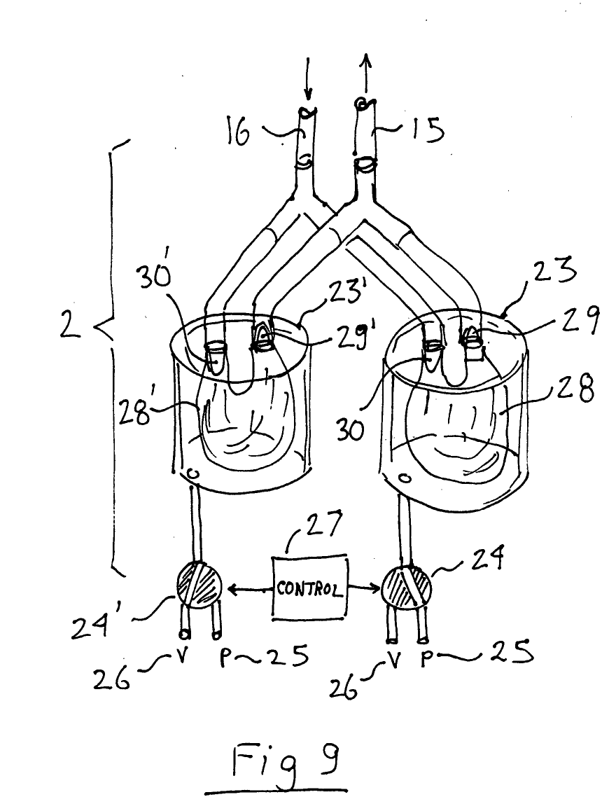

At the point of entry into the body the tubes 15′ are reduced in diameter, which also reduces the diameter of the inside lumen 16. Since the pressure tube is no longer a single tube, a manifold 21 can be used to convert it to a single tube connected to pump 2. At both ends of the catheter, where tubes 15′ do not continue, a thin flexible tube 16′ has to be added. FIG. 8 shows further detail of the distal end, normally inserted into the left ventricle. Tube 16 is slightly reinforced to withstand vacuum. Since the length of tube 16′ is about 10% of the total catheter length, the vacuum on tube 16′ is also about 10% of the total vacuum used, or about 50 mmHg. Such a low vacuum needs minimal reinforcement. The Nitinol wire used to form cage 19 can be continued as a large pitch spiral 22. It is desired to keep the pitch of the spiral large in order to make tube 16′ collapsible, to match the insertion diameter of the catheter. Obviously only the parts entering the body need to be collapsible. When the catheter is used to assist the right ventricle, the pressure part extends beyond the suction part, making the design simpler and eliminating the need for reinforcement 22. Instead, delivery ports 17 are at the end of the catheter and suction holes are located between tubes 15′ at some point before the end of the catheter. By the way of example, tube 15 was made from 12 tubes each one 2 mm diameter and about 0.03 mm wall thickness bonded together to form a round tubing with an internal lumen of about 7 mm diameter, as shown in FIG. 7A. At the point the catheter enters the body all tubes were heat shrunk to a diameter of about 0.8 mm for a length of 10 mm. At this point the diameter of the inner lumen was about 3.5 mm and the entrance hole required in the artery was about 15 F (5 mm). Entrance holes as small as 12 F can be used if more hemolysis can be tolerated (i.e. for shorter periods of use). It is convenient to use stent balloon tubing as it is heat shrinkable and can withstand large pressure. Such tubing is available from Advanced Polymers (www.advpoly.com) in a wide range of diameters and wall thickness. The tubes were bonded using Silicone adhesive. This design has a fixed length from the distal tip to the entrance hole in the artery. The suction section 16 inside the heart was also made from similar tubing reinforced by a 0.3 mm Nitinol wire, which also forms the cage, as shown in FIG. 8. When expanded section 16 reached about 7 mm in diameter and can be collapsed to below 4 mm. The tubing outside the body can be regular flexible PVC tubes. For a flow of 5 l/min, the suction used was about 650 mmHg and the pressure 1500 mmHg. It is best to fill the pump with standard medical saline solution before starting. This prevents air bubbles and allows the pressure side to inflate before vacuum is applied. A manual vacuum release valve can also be used to assist starting. The valve is closed as soon as pressure was established in the pressure tubes.

A different type of blood pump, with low levels of hemolysis (blood damage), is shown in FIG. 9. It is known that the red blood cells are damaged by high turbulence areas and high shear flow. The pump in FIG. 9 has pure laminar flow and the disposable part is low cost: just two plastic bags similar to the bags used to store blood. Since such pumps generate pulsatile flow by alternating between suction and pressure, two units are used with opposite timing to generate continuous flow or partially pulsatile flow which can be synchronized to the EKG of the patient. The pump comprises of two containers 23 and 23′ which can be easily opened to insert bags 28 and 28′. The containers can be transparent for ease of monitoring. Each container can be connected to a source of air pressure 25 or vacuum 26 using valves 24 and 24′ controlled by controller 27. When valve 24 is set for pressure, valve 24′ is set for vacuum. Bags 28 and 28′ expand and contract in response to the alternating pressure and vacuum. The output side of both bags is connected together to pressure tube 15 and suction sides are connected together to suction tube 16. Inside the bags flaps 29 and 29′ form output valves while flaps 30 and 30′ form input valves. The valves operate in a similar fashion to a cardiac mitral valve. The flaps can be made from the same material as the bag. The disposable part comprises of the catheter and the two bags. Since the bags and valves are large all the flow is laminar with low Reynolds numbers.

Beneficial surface treatments for reduction of clotting may be used on the surfaces coming in contact with the blood. Coatings can be anticoagulants, such as heparin, or special surface modifications. It was found out that a superhydrophobic surface can reduce or eliminate clotting. A superhydrophobic surface is a hydrophobic surface having a contact angle approaching 180 degrees with a drop of water. Such surfaces can be created by microscopic texturing with polymer or inorganic “hairs” having a diameter significantly less than one micron. The art of superhydrophobic surfaces is well known. It was also found out that texturing on a more coarse scale can decrease or eliminate clotting. Any combination of the above methods can be used, for example texturing treated with a hydrophobic agent such as a fluorocarbon or silicone.

While the disclosure details, by the way of example, a cardiac assist application, the invention can find many other uses in delivering liquids into a body lumen. In its simplest form a single sleeve is used, without an inner tube. Such a sleeve can be beneficial in procedures such as dialysis or when delivering fluids to the intestinal or urinary system. In all these cases it can deliver a larger amount of fluid through a given opening compared to a constant diameter catheter. Since the catheter will expand to a large diameter over most of its length inside the body, the main flow restriction will be at the entry point to the body. Because of the short restriction length and smooth transition it can be overcome by increasing the pressure of the pump. For fluids that can tolerate high pressures the flow improvement can be dramatic.

The word “catheter” in this disclosure should be interpreted broadly as any device inserted into the human body.

1. A fluid delivery catheter for insertion into a body lumen, said catheter expandable along most of its length inside the body from a smaller diameter used during insertion to a larger diameter by the pressure of said fluid, said expansion being smaller at the point said catheter is inserted into said lumen than at other points of expansion.

2. A cardiac assist device comprising a blood delivery catheter and a pump, said catheter having a highly flexible sleeve surrounding a less flexible inner tube, said inner tube used for suction and said sleeve expanding along most of its length when pressurized by the blood.

3. A multi-lumen catheter for the delivery of fluids into the body having at least one suction lumen and one pressure lumen and in which the ability of said suction lumen to withstand vacuum is created by pressurizing said pressure lumen.

4. A multi lumen catheter as in claim 3 wherein the wall of said suction lumen is formed by a plurality of pressure lumens bonded together along their length.

5. A catheter as in claim 3 wherein said suction lumen is further divided by partitions to reduce turbulence.

6. A catheter as in claim 1 wherein inside of said catheter is further divided by partitions to reduce turbulence.

7. A catheter as in claim 3 wherein said suction is between −300 to −700 mmHg and said pressure is between 500 to 2500 mmHg.

8. A catheter as in claim 3 wherein said pressure and suction are created by a peristaltic pump.

9. A catheter as in claim 3 wherein said pressure and suction are created by a centrifugal pump.

10. A catheter as in claim 3 wherein said pressure and suction are created by a pump exposing a bag to pressure and vacuum, said bag and said catheter are disposable.

11. A catheter as in claim 3 wherein said pressure and suction are created by a pump exposing a bag to pressure and vacuum, said bag equipped with valves made of flexible flaps.

12. A catheter as in claim 1 wherein the surfaces in touch with the fluid are coated by a superhydrophobic coating.

13. A catheter as in claim 1 wherein the surfaces in touch with the fluid are coated by an anti-coagulant.

14. A catheter as in claim 3 wherein the surfaces in touch with the fluid are textured.

15. A catheter as in claim 3 wherein the surfaces in touch with the fluid are coated by a superhydrophobic coating.

16. A catheter as in claim 3 wherein the surfaces in touch with the fluid are coated by an anti-coagulant.

17. A catheter as in claim 2 wherein the outside surface of said inner tube is textured in order to allow blood flow when said surrounding sleeve is compressed against said inner tube.

18. A catheter as in claim 3 wherein the suction lumen is inserted into a part of the heart and comprises of a collapsible structure.

19. A catheter as in claim 1 wherein the outside diameter of the catheter at the point of insertion is between 3 to 8 mm and the diameter of the catheter inside the body is between 5 and 15 mm.

20. A catheter as in claim 3 comprising of a short section with a reduced outside diameter after being pressurized.
